(19) United States
(12) Patent Application Publication
(10) Pub. No.: US 2020/0330508 A1
(43) Pub. Date: Oct. 22, 2020

(54) COMPOSITIONS FOR PREVENTION OR SUPPRESSION OF CANCER OR CANCER RELAPSE

(71) Applicants: FU NAN WANG, HACIENDA HEIGHTS, CA (US); MING CHU HSU, HACIENDA HEIGHTS, CA (US); SEBO DIH WANG, HACIENDA HEIGHTS, CA (US); SEBO LING WANG, HACIENDA HEIGHTS, CA (US); SEBO MICHELLE WANG, HACIENDA HEIGHTS, CA (US); SEBO GENE WANG, HACIENDA HEIGHTS, CA (US)

(72) Inventors: FU NAN WANG, HACIENDA HEIGHTS, CA (US); MING CHU HSU, HACIENDA HEIGHTS, CA (US); SEBO DIH WANG, HACIENDA HEIGHTS, CA (US); SEBO LING WANG, HACIENDA HEIGHTS, CA (US); SEBO MICHELLE WANG, HACIENDA HEIGHTS, CA (US); SEBO GENE WANG, HACIENDA HEIGHTS, CA (US)

(21) Appl. No.: 16/388,104

(22) Filed: Apr. 18, 2019

(51) Int. Cl. A61K 33/30; A61K 33/04; A61K 33/06; A61K 33/26; A61K 33/18; A23L 33/16; A61K 9/00; A61P 35/00

(52) U.S. Cl. CPC A61K 33/30; A61K 33/04; A61K 33/06; A61P 35/00; A61K 33/18; A23L 33/16; A61K 9/0053; A61K 33/26

(57) ABSTRACT

The present invention relates to a rare element composition for preventing or suppressing a cancer or cancer relapse. The composition comprises selenium, molybdenum, zinc, germanium, magnesium, iron and iodine. Also provided is a method for preventing or suppressing a cancer or a cancer relapse, comprising administering the composition of the invention to a subject.

### FIELD OF THE INVENTION

The present invention relates to a rare element composition for preventing or suppressing a cancer or cancer relapse.

### BACKGROUND OF THE INVENTION

Cancer is a group of diseases that involves unregulated cell growth. Cancer is usually treated with chemotherapy, radiation therapy, and/or surgery. In recent years, modulation of the tumor microenvironment is becoming an increasingly popular research topic in the field of immunotherapy, and antibody therapy directed against certain negative immunologic regulators (immune checkpoints) has been shown to be successful as an anti-tumor treatment in several cancer types. However, survival rates vary by the type and location of the cancer and the extent of the disease.

Cancer rates are currently rising, partly due to increased life expectancies throughout the world. Cancer prevention is action taken to lower the risk of getting cancer. According to WHO's website (https://www.who.int/cancer/prevention/en/), between 30-50% of all cancer cases are preventable. Prevention offers the most cost-effective long-term strategy for the control of cancer. National policies and programmes should be implemented to raise awareness, to reduce exposure to cancer risk factors and to ensure that people are provided with the information and support they need to adopt healthy lifestyles. U.S. Pat. No. 10,117,902 provides nutritional compositions that are employed as oral supplementation to the human diet, which is for supplementation to the diet of the cancer patient, as well as preventative dietary supplementation aimed at supporting the human immune system for those not currently suffering from cancer.

### SUMMARY OF THE INVENTION

The present disclosure provides a composition comprising selenium, molybdenum, zinc, germanium, magnesium, iron and iodine. Certain embodiment of the present disclosure includes the selenium, molybdenum, zinc, germanium, magnesium, iron and iodine in the amounts ranging from about 100 mcg to about 200 mcg, about 10 mg to about 20 mg, about 11 mg to about 22 mg, about 150 mg to about 300 mg, about 200 mg to about 400 mg, about 20 mg to about 40 mg and about 150 mcg to about 300 mcg, respectively. In some embodiments, the compositions of the present disclosure are formulated into pharmaceutical compositions or dietary supplements by further combining with a pharmaceutically or food acceptable carrier.

The present disclosure also provides a method for preventing or suppressing a cancer or a cancer relapse, comprising administering the composition of the present disclosure to a subject. In one embodiment, the composition of the present disclosure is administered once a day. In one embodiment, the composition of the present disclosure is administered at a daily dosage ranging from about 350 mg to about 800 mg or about 5 mg/kg to about 15 mg/kg body weight.

Certain embodiments include that Se, Mo, Zn, Ge, Mg, Fe and I are in its element form or their organocompound form.

In one embodiment, the composition and method of the present disclosure is beneficial to prevent cancers in a subject.

The composition and method of the present disclosure can suppress, reduce the severity, reduce the risk, or prevent or inhibit a cancer in a subject. In one embodiment, the subject has been previously treated with chemotherapy, radiotherapy or biological therapy.

### DETAILED DESCRIPTION OF THE INVENTION

Terms not specifically defined herein should be understood according to the meanings that would be given to them by one of skill in the art in light of the disclosure and the context. As used in the specification, however, unless specified to the contrary, the following terms have the meaning indicated according to the following conventions.

The singular forms “a,” “an,” and “the” include plural referents unless the context clearly dictates otherwise

As used herein, the term “preventing” or “prevention” refers to preventing the disease; for example, preventing a disease, condition or disorder in an individual who may be predisposed to the disease, condition or disorder but does not yet experience or display the pathology or symptomatology of the disease.

As used herein, the term “preventive effective amount” means an amount of an agent that (i) prevents the particular disease or condition, (ii) attenuates, ameliorates, or eliminates one or more symptoms of the particular disease or condition, or (iii) prevents or delays the onset of one or more symptoms of the particular disease or condition described herein.

In one aspect, the present disclosure provides a composition comprising selenium, molybdenum, zinc, germanium, magnesium, iron and iodine. In one embodiment, the amounts of selenium, molybdenum, zinc, germanium, magnesium, iron and iodine range from about 100 mcg to about 200 mcg, about 10 mg to about 20 mg, about 11 mg to about 22 mg, about 150 mg to about 300 mg, about 200 mg to about 400 mg, about 20 mg to about 40 mg and about 150 mcg to about 300 mcg, respectively.

In some embodiments, the amount of selenium ranges from about 100 mcg to about 180 mcg, about 100 mcg to about 160 mcg, about 100 mcg to about 140 mcg, about 100 mcg to about 120 mcg, about 120 mcg to about 200 mcg, about 120 mcg to about 180 mcg, about 120 mcg to about 160 mcg, about 120 mcg to about 140 mcg, about 140 mcg to about 200 mcg, about 140 mcg to about 180 mcg or about 140 mcg to about 160 mcg.

In some embodiments, the amount of molybdenum ranges from about 10 mg to about 18 mg, about 10 mg to about 16 mg, about 10 mg to about 14 mg, about 10 mg to about 12 mg, about 12 mg to about 20 mg, about 12 mg to about 18 mg, about 12 mg to about 16 mg, about 12 mg to about 14 mg, about 14 mg to about 20 mg, about 14 mg to about 18 mg, about 14 mg to about 16 mg, about 16 mg to about 20 mg or about 16 mg to about 18 mg.

In some embodiments, the amount of zinc ranges from about 11 mg to about 20 mg, about 11 mg to about 18 mg, about 11 mg to about 16 mg, about 11 mg to about 14 mg, about 13 mg to about 22 mg, about 13 mg to about 20 mg, about 13 mg to about 18 mg, about 13 mg to about 16 mg, about 15 mg to about 22 mg, about 15 mg to about 20 mg, about 15 mg to about 18 mg, about 17 mg to about 22 mg or about 19 mg to about 22 mg.

In some embodiments, the amount of germanium ranges from about 150 mg to about 250 mg, about 150 mg to about 200 mg, about 200 mg to about 300 mg or about 250 mg to about 300 mg.

In some embodiments, the amount of magnesium ranges from about 200 mg to about 350 mg, about 200 mg to about 300 mg, about 200 mg to about 250 mg, about 250 mg to about 400 mg, about 250 mg to about 350 mg, about 250 mg to about 300 mg, about 300 mg to about 400 mg or about 350 mg to about 400 mg.

In some embodiments, the amount of iron ranges from about 20 mg to about 35 mg, about 20 mg to about 30 mg, about 20 mg to about 25 mg, about 25 mg to about 40 mg, about 25 mg to about 35 mg, about 25 mg to about 30 mg, about 30 mg to about 40 mg or about 35 mg to about 40 mg.

In some embodiments, the amount of iodine ranges from about 150 mcg to about 250 mcg, about 150 mcg to about 200 mcg, about 200 mcg to about 300 mcg, about 200 mcg to about 250 mcg or about 250 mcg to about 300 mcg.

In another aspect, the present disclosure provides a method for preventing or suppressing a cancer or a cancer relapse, comprising administering the composition of the present disclosure to a subject. In one embodiment, the composition of the present disclosure is administered once a day. In one embodiment, the composition of the present disclosure is administered at a dosage ranging from about 350 mg to about 800 mg or about 5 mg/kg to about 15 mg/kg body weight. In one embodiment, the dosage is a daily dose.

In some embodiments, the composition of the present disclosure is administered at a dosage ranging from about 400 mg to about 800 mg, about 450 mg to about 800 mg, about 500 mg to about 800 mg, about 550 mg to about 800 mg, about 600 mg to about 800 mg, about 650 mg to about 800 mg, about 700 mg to about 800 mg, about 750 mg to about 800 mg, about 350 mg to about 700 mg, about 350 mg to about 650 mg, about 350 mg to about 600 mg, about 350 mg to about 550 mg, about 350 mg to about 500 mg, about 350 mg to about 450 mg, about 350 mg to about 400 mg, about 400 mg to about 800 mg, about 450 mg to about 800 mg, about 500 mg to about 800 mg, about 550 mg to about 800 mg, about 600 mg to about 800 mg, about 650 mg to about 800 mg, about 700 mg to about 800 mg, about 750 mg to about 800 mg, about 400 mg to about 750 mg, about 400 mg to about 700 mg, about 400 mg to about 650 mg, about 400 mg to about 600 mg, about 400 mg to about 550 mg, about 400 mg to about 500 mg, about 400 mg to about 450 mg, about 450 mg to about 800 mg, about 450 mg to about 750 mg, about 450 mg to about 700 mg, about 450 mg to about 650 mg, about 450 mg to about 600 mg, about 450 mg to about 550 mg, about 450 mg to about 500 mg, about 500 mg to about 800 mg, about 500 mg to about 700 mg, about 500 mg to about 650 mg, about 500 mg to about 600 mg, about 500 mg to about 550 mg, about 550 mg to about 800 mg, about 550 mg to about 750 mg, about 550 mg to about 700 mg, about 550 mg to about 650 mg, about 550 mg to about 600 mg, about 600 mg to about 800 mg, about 600 mg to about 750 mg, about 600 mg to about 700 mg or about 600 mg to about 650 mg. In a further embodiment, the dosage ranging from about 391.25 mg to about 782.5 mg. In one embodiment, the dosage is a daily dose.

In one embodiment, the composition of the present disclosure is administered at a dosage ranging from about 5 mg/kg to 12 mg/kg body weight, about 5 mg/kg to 10 mg/kg body weight, about 5 mg/kg to 8 mg/kg body weight, about 7 mg/kg to 12 mg/kg body weight, about 7 mg/kg to 10 mg/kg body weight or about 9 mg/kg to 12 mg/kg body weight. In a further embodiment, the dosage ranging from about about 6.02 mg/kg to 12.04 mg/kg body weigh. In one embodiment, the dosage is a daily dose.

Selenium is a chemical element with symbol Se, which is an essential micronutrient for animals. Selenium is a component of the unusual amino acids selenocysteine and selenomethionine. In humans, selenium is a trace element nutrient that functions as cofactor for reduction of antioxidant enzymes, such as glutathione peroxidases and certain forms of thioredoxin reductase found in animals. Dietary selenium comes from nuts, cereals and mushrooms. In one embodiment, the selenium occurs in an element Se or organoselenium compounds.

Molybdenum is a chemical element with symbol Mo, which is an essential trace dietary element. Four mammalian Mo-dependent enzymes are known, all of them harboring a pterin-based molybdenum cofactor (Moco) in their active site: sulfite oxidase, xanthine oxidoreductase, aldehyde oxidase, and mitochondrial amidoxime reductase. People severely deficient in molybdenum have poorly functioning sulfite oxidase and are prone to toxic reactions to sulfites in foods. Dietary sources of MO include green beans, eggs, sunflower seeds, wheat flour, lentils, cucumbers, and cereal grain. In one embodiment, the molybdenum occurs in an element Mo or organomolybdenum compounds.

Zinc is a chemical element with symbol Zn, which is an essential trace element for animals. Zinc is required for the function of over 300 enzymes and 1000 transcription factors, and is stored and transferred in metallothioneins. Animal products such as meat, fish, shellfish, fowl, eggs, and dairy contain zinc. The food plants that contain the most zinc are wheat (germ and bran) and various seeds, including sesame, poppy, alfalfa, celery, and mustard. Zinc is also found in beans, nuts, almonds, whole grains, pumpkin seeds, sunflower seeds, and blackcurrant. In one embodiment, the zinc occurs in an element Zn or organozinc compounds.

Germanium is a chemical element with symbol Ge. Germanium is not thought to be an essential element for any living organism. Some complex organic germanium compounds are being investigated as possible pharmaceuticals. In one embodiment, the germanium occurs in an element Ge or organogermanium compounds.

Magnesium is a chemical element with symbol Mg, which essential to the basic nucleic acid chemistry of all cells of all known living organisms. More than 300 enzymes require magnesium ions for their catalytic action, including all enzymes using or synthesizing ATP and those that use other nucleotides to synthesize DNA and RNA. Spices, nuts, cereals, cocoa and vegetables are rich sources of magnesium. Green leafy vegetables such as spinach are also rich in magnesium. In one embodiment, the magnesium occurs in an element Mg or organomagnesium compounds.

Iron is a chemical element with symbol Fe, which is required for life. The iron sulfur clusters are pervasive and include nitrogenase, the enzymes responsible for biological nitrogen fixation. Iron-containing proteins participate in transport, storage and used of oxygen. Iron proteins are involved in electron transfer. Rich sources of dietary iron include red meat, oysters, lentils, beans, poultry, fish, leaf vegetables, watercress, tofu, chickpeas, black-eyed peas, and blackstrap molasses. In one embodiment, the iron occurs in an element Fe or organoiron compounds.

Iodine is a chemical element with symbol I, which is an essential element for life. It is required for the synthesis of the growth-regulating thyroid hormones thyroxine and triiodothyronine. Natural sources of dietary iodine include seafood, such as fish, seaweeds (such as kelp) and shellfish, dairy products and eggs. In one embodiment, the iodine occurs in an element I or organoiodine compounds.

The present disclosure combines the elements, Se, Mo, Zn, Ge, Mg, Fe and I or their organocompounds to form a composition. The composition of the present disclosure is beneficial to prevent cancers in a subject. In some embodiments, the cancer is liver cancer, lung cancer, gastric cancer, esophageal cancer, breast cancer, prostate cancer, adrenocortical carcinoma, anal cancer, bladder cancer, brain tumor, glioma, cervical cancer, colon cancer, central nervous system (CNS) cancer, endometrial cancer, esophageal cancer, extrahepatic bile duct cancer, eye cancer, intraocular melanoma, gallbladder cancer, head and neck cancer, hypopharyngeal cancer, islet cell carcinoma, laryngeal cancer, leukemia, acute lymphoblastic, leukemia, oral cavity cancer, non-small cell lung cancer, small cell, lymphoma, central nervous system (primary), lymphoma, cutaneous T-cell, lymphoma, Hodgkin's disease, non-Hodgkin's disease, malignant mesothelioma, melanoma, metastic squamous carcinoma, multiple myeloma, plasma cell neoplasms, myelodysplastic syndrome, myeloproliferative disorders, nasopharyngeal cancer, osteosarcoma, ovarian cancer, pancreatic cancer, exocrine, islet cell carcinoma, rectal cancer, renal cancer, renal cell cancer, skin cancer, melanoma, small intestine cancer, thyroid cancer, urethral cancer, uterine cancer, sarcoma, vaginal cancer, vulvar cancer, or any combination thereof. Preferably, the cancer is liver cancer, lung cancer, gastric cancer, esophageal cancer, breast cancer or prostate cancer.

In one embodiment, the present disclosure is directed to a method of suppressing, reducing the severity, reducing the risk, or preventing or inhibiting cancer in a subject. In one embodiment, the subject has been previously treated with chemotherapy, radiotherapy or biological therapy. In one embodiment, “chemotherapy” refers to chemical treatment for cancer such as drugs that kill cancer cells directly. In one embodiment, “radiotherapy” refers to high energy x-rays and similar rays (such as electrons) to treat disease. In one embodiment “biological therapy” refers to substances that occur naturally in the body to destroy cancer cells.

The compositions of the present disclosure are formulated into pharmaceutical compositions or dietary supplements by further combining with a pharmaceutically or food acceptable carrier. The term “pharmaceutically or food acceptable carrier” refers to any suitable adjuvants, carriers, excipients, or stabilizers, and can be in solid or liquid form such as, tablets, capsules, powders, pills, dragees, solutions, suspensions, gels, syrups, or emulsions. Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art.

The compositions of the present disclosure may be orally administered, for example, with an inert diluent, or with an assimilable edible carrier, or they can be enclosed in hard or soft shell capsules, or they can be compressed into tablets, or they can be incorporated directly with the food of the diet.

In one embodiment, the compositions of the present disclosure are administered in combination with an anti-cancer agent or an anti-cancer therapy.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention.

### EXAMPLES

### Human Study

The criteria for inclusion of subjects are as follows.
1. Male and female subjects 55-60 (inclusive) years of age.
2. Normal life style.
3. No smoking and alcohols.
4. No gastrointestinal tract or respiratory tract or other disorders.
5. No genetic or hereditary familial cancer history.
6. No mental health problems.
7. Tumor markers (CEA, CA125, CA 19-9, AFP, PSA, CA 15-3, CYFRA 21-1, Neuron-specific enolase, squamous cell carcinoma antigen, TPA, Beta-2-microglobulin and human chorionic gonadotropin) are within normal ranges.
8. Pap Smear Test: Normal.
9. Hematology Test, Urine Test, Liver Function Test, Renal Function Test, Hormone Test (T4, TSH, prolactin, estrogen and testosterone), Occult blood: Normal.
10. Mammography: Normal.

107 subjects (62 are males and 43 are females) were enrolled to attend a human study. The subjects were divided to two groups, experimental group (42 subjects) and control group (65 subjects). The subjects in experimental group received the composition of the present disclosure once a day from March 2010 to July 2018. In the experimental group, no subject suffered from cancer within the study period, whereas three subjects in the control group suffered cancer (one suffered from lung adenocarcinoma, and the other two subjects suffered from prostate cancer and colon cancer respectively).

What is claimed is:

1. A composition for preventing or suppressing a cancer or cancer relapse, comprising selenium, molybdenum, zinc, germanium, magnesium, iron and iodine.

2. The composition of claim 1, wherein the amounts of selenium, molybdenum, zinc, germanium, magnesium, iron and iodine range from about 100 mcg to about 200 mcg, about 10 mg to about 20 mg, about 11 mg to about 22 mg, about 150 mg to about 300 mg, about 200 mg to about 400 mg, about 20 mg to about 40 mg and about 150 mcg to about 300 mcg, respectively.

3. The composition of claim 1, wherein selenium, molybdenum, zinc, germanium, magnesium, iron and iodine are in the element form or organocompound form.

4. The composition of claim 1, which is formulated into a pharmaceutical composition or a dietary supplement.

5. The composition of claim 4, which is in solid or liquid form.

6. The composition of claim 4, which is in the form of tablet, capsule, powder, pill, dragee, solution, suspension, gel, syrup, or emulsion.

7. The composition of claim 1, wherein the cancer is liver cancer, lung cancer, gastric cancer, esophageal cancer, breast cancer or prostate cancer.

8. A method for preventing or suppressing a cancer or a cancer relapse, comprising administering the composition of claim 2 to a subject.

9. The method of claim 8, wherein the composition is administered once a day.

10. The method of claim 8, wherein the composition is administered at a dosage ranging from about 350 mg to about 800 mg or about 5 mg/kg to about 15 mg/kg body weight.

11. The method of claim 8, wherein the composition is orally administered.

12. The method of claim 8, wherein the composition is a pharmaceutical composition or a dietary supplement

13. The method of claim 8, wherein the cancer is liver cancer, lung cancer, gastric cancer, esophageal cancer, breast cancer or prostate cancer.

14. The method of claim 8, wherein the administration of the composition can suppress, reduce the severity, reduce the risk, or prevent or inhibit cancer in a subject.

15. The method of claim 8, wherein the subject has been previously treated with chemotherapy, radiotherapy or biological therapy.

16. The method of claim 8, wherein the composition of claim 2 is administered or treated in combination with an anti-cancer agent or an anti-cancer therapy.
